(19) United States
(12) Patent Application Publication
(10) Pub. No.: US 2012/0222333 A1
(43) Pub. Date: Sep. 6, 2012

(54) VIBRATING ORTHOTIC SHOE INSERT AND METHOD OF MANUFACTURING THE SAME

(75) Inventors: Timothy J. Short, Tucson, AZ (US); John P. Powers, Tucson, AZ (US)

(73) Assignee: PARADOCS, LLC, Tucsan, AZ (US)

(21) Appl. No.: 13/041,333

(22) Filed: Mar. 4, 2011

(51) Int. Cl. A61F 5/14; A43B 7/14; A61H 1/00

(52) U.S. Cl. 36/140; 601/46; 12/146 M

(57) ABSTRACT

Vibrating orthotic shoe inserts and a method of manufacturing the same. In an embodiment, a vibrating orthotic shoe insert includes a forefoot portion configured to support a forefoot portion of a human foot; an arch portion configured to support an arch of the human foot, the arch portion including an area configured to be under a porta pedis of the human foot; and a heel portion configured to support a heel of the human foot. The arch portion includes a vibration element positioned within the area configured to be under the porta pedis of the human foot. The vibration element is configured to deliver vibration to the porta pedis of the human foot.

### BACKGROUND

1. Field

Embodiments of the present invention relate generally to orthotics. More particularly, embodiments of the present invention relate to a vibrating orthotic shoe insert and method of manufacturing the same.

2. Description of the Related Art

A common podiatry complaint is foot pain such as chronic foot pain. Such foot pain can be caused by a variety of conditions. For example, nerve conditions such as peripheral neuropathy and chronic regional pain syndrome are but a few of many causes of foot pain. Common protocols for addressing foot pain include drug therapy and surgery. These protocols may have some positive effect, however; they also may have side effects. For example, drug therapy may cause medical side effects. Further, drug cost may be an issue for a patient. Surgery necessarily entails at least some risk, and may also present cost issues for a patient.

Orthotic shoe inserts are widely known in the art. Such orthotic shoe inserts may be used for a variety of purposes to treat a variety of conditions. For example, orthotic shoe inserts may be used to correct an abnormal walking pattern or to correct a collapsed arch. Such orthotic shoe inserts may be custom molded to fit a patient's foot or prefabricated. Such orthotic shoe inserts may also provide some pain relief by providing cushioning or by supporting a patient's foot in a proper position. However, such orthotic shoe inserts may not achieve an acceptable level of pain relief for pain associated with certain foot conditions.

Various vibrating foot devices are known in the art. Such vibrating foot devices may be used for a variety of purposes to treat a variety of conditions. For example, a vibrating foot massage insole has been disclosed for providing a vibrating massage to a user's foot. As another example, a shoe with a vibrating sole has been disclosed for improving a user's balance. The vibration may be so slight as to be undetectable. Such vibrating foot devices may also provide some pain relief by, for example, providing a vibrating massage. However, such vibrating foot devices may not achieve an acceptable level of pain relief for pain associated with certain foot conditions.

In light of the foregoing, it is desirable to provide a new protocol for achieving an acceptable level of pain relief for pain associated with certain foot conditions.

### BRIEF SUMMARY

According to an aspect of the invention, a vibrating orthotic shoe insert is provided. The vibrating orthotic shoe insert includes a forefoot portion configured to support a forefoot portion of a human foot; an arch portion configured to support an arch of the human foot, the arch portion including an area configured to be under a porta pedis of the human foot; and a heel portion configured to support a heel of the human foot. The arch portion includes a vibration element positioned within the area configured to be under the porta pedis of the human foot. The vibration element is configured to deliver vibration to the porta pedis of the human foot.

According to another aspect of the invention, a method of manufacturing a vibrating orthotic shoe insert is provided. The method comprises setting dimensions of the vibrating orthotic shoe insert; and forming the vibrating orthotic shoe insert according to the set dimensions. The vibrating orthotic shoe insert includes a forefoot portion configured to support a forefoot portion of a human foot; an arch portion configured to support an arch of the human foot, the arch portion including an area configured to be under a porta pedis of the human foot; and a heel portion configured to support a heel of the human foot. The arch portion includes a vibration element positioned within the area configured to be under the porta pedis of the human foot. The vibration element is configured to deliver vibration to the porta pedis of the human foot.

According to another aspect of the invention, a vibrating orthotic shoe insert is provided. The vibrating orthotic shoe insert includes a forefoot portion including a top surface configured to support a forefoot portion of a human foot; an arch portion being formed of an arch fill material and including: a top surface configured to support an arch of the human foot; an area configured to be under a porta pedis of the human foot; and at least a sub-portion of the arch portion located above the area configured to be under the porta pedis of the human foot, the at least a sub-portion being formed of an arch fill material of a high density, a heel portion including a top surface configured to support a heel of the human foot, the heal portion being formed of a high density material; a power source positioned within the heal portion; and a vibration element within the area configured to be under the porta pedis of the human foot within the arch portion. The vibration element is configured to receive power from the power source. The vibration element is configured to deliver vibration to the porta pedis of the human foot through the at least a sub-portion of the arch portion.

The foregoing and other aspects will become apparent from the following detailed description when considered in conjunction with the accompanying drawing figures.

### BRIEF DESCRIPTION OF THE DRAWINGS

FIG. 1 is a top view cut-away of a vibrating orthotic shoe insert according to an embodiment of the present invention.

FIG. 2 is a side view cut-away of the vibrating orthotic shoe insert of FIG. 1.

FIG. 3 is a front view cut-away of the vibrating orthotic shoe insert of FIG. 1.

FIG. 4 is a schematic representation of a method for manufacturing a vibrating orthotic shoe insert according to an embodiment of the present invention.

### DETAILED DESCRIPTION

Reference will now be made in detail to embodiments of the present invention, examples of which are illustrated in the accompanying drawings, wherein like reference numerals refer to the like elements throughout. The embodiments are described below to explain the present invention by referring to the figures.

“As used in the description of this application, the terms “a”, “an” and “the” may refer to one or more than one of an element (e.g., item or act). Similarly, a particular quantity of an element may be described or shown while the actual quantity of the element may differ. The terms “and” and “or” may be used in the conjunctive or disjunctive sense and will generally be understood to be equivalent to “and/or”. Elements from an embodiment may be combined with elements of another. No element used in the description of this application should be construed as critical or essential to the invention unless explicitly described as such. Further, when an element is described as “connected,” “coupled,” or otherwise linked to another element, it may be directly linked to the other element, or intervening elements may be present.

According to an embodiment of the present invention, a vibrating orthotic shoe insert may be provided. The vibrating orthotic shoe insert may be placed in a patient's shoe and support the patient's foot. The vibrating orthotic shoe insert may include a vibration element positioned below the porta pedis of the patient's foot. When power is supplied to the vibration element, the vibration element may deliver (directly or indirectly) detectable vibration to the porta pedis of the patient's foot. The vibration delivered to the porta pedis of the patient's foot may overload the nerves passing through the porta pedis thereby interrupting pain signals. This interruption of pain signals may thereby provide pain management for the patient.

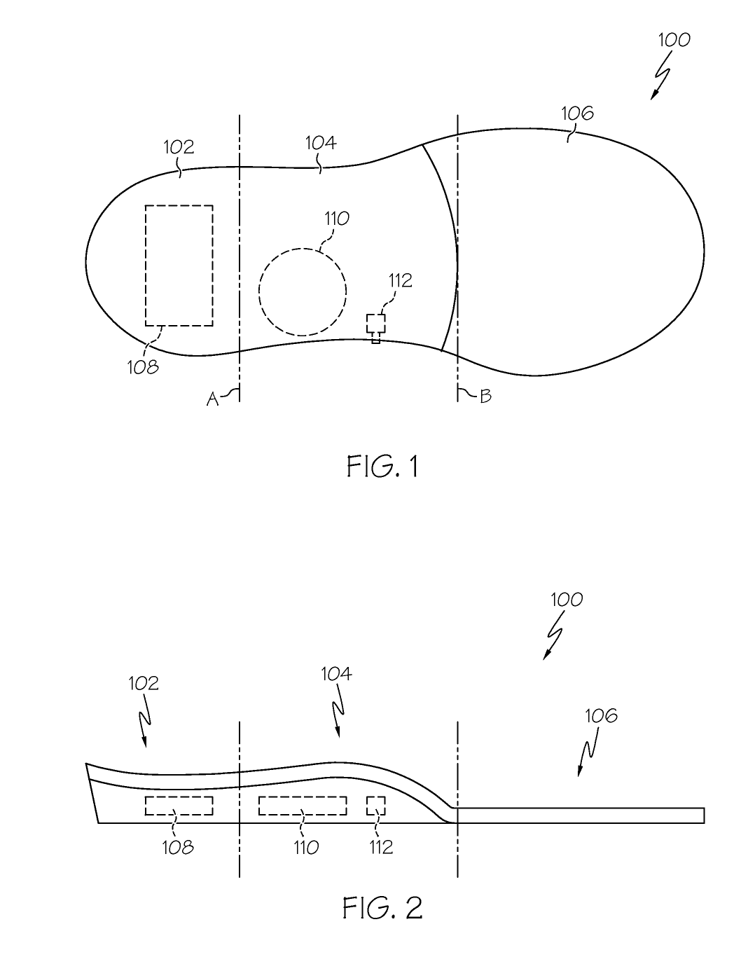

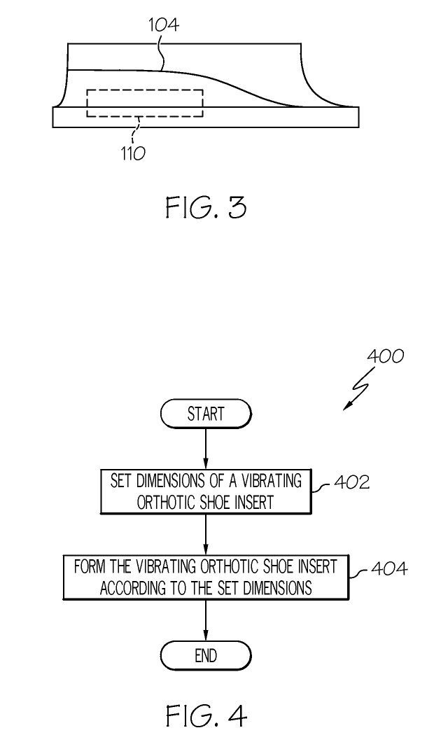

FIG. 1 is a top view cut-away of a vibrating orthotic shoe insert 100 according to an embodiment of the present invention. FIG. 2 is a side view cut-away of the vibrating orthotic shoe insert 100 of FIG. 1. FIG. 3 is a front view cut-away of the vibrating orthotic 100 shoe insert of FIG. 1.

As shown in FIGS. 1 and 2, the vibrating orthotic shoe insert 100 may include a heel portion 102, an arch portion 104, and a forefoot portion 106. The vibrating orthotic shoe insert 100 may further include a power source 108, a vibration element 110, and a switch 112. As shown, the heel portion 102 and the arch portion 104 may be logically divided by line A. Similarly, the arch portion 104 and the forefoot portion 106 may be logically divided by line B. However, although the heel, arch, and forefoot portions are shown along with specific divisions there between, the invention is not so limited. For example, in an alternative embodiment, a vibrating orthotic shoe insert including a heel portion and an arch portion may be provided. In such an embodiment, the specific division point between the heel portion and the arch portion may additionally be different than that shown in FIG. 1.

The vibrating orthotic shoe insert 100 may be covered by a shell. For example, the vibrating orthotic shoe insert may be covered by a polyethylene shell layer. However, the invention is not so limited. For example, in an alternative embodiment, a vibrating orthotic shoe insert may be covered by a fiberglass shell. The shell of the vibrating orthotic shoe insert 100 may serve as a water resistant or water proof feature. The dimensions of the vibrating orthotic shoe insert may vary depending on the patient's size, pathology, foot-type, and foot dimensions. Similarly, the materials of the vibrating orthotic shoe insert, although specifically discussed below, may vary depending on the patient's size, pathology, foot-type, and foot dimensions.

The heel portion 102 of the vibrating orthotic shoe insert may also be referred to as a rearfoot post. The heel portion 102 may be formed out of a high density material. For, example, in the embodiment shown, the heel portion 102 may be formed of high density foam, such asethylene vinyl acetate. However, the invention is not so limited. For example, in an alternative embodiment, a heel portion may be formed of hard rubber. The heel portion 102 may be configured to support the heel of a human foot (e.g., a patient's foot). The top surface of the heel portion may be slightly contoured so as to correspond with the shape of the patient's foot. Accordingly, the top surface of the heel portion 102 may correctly position the patient's foot on the orthotic shoe insert. However, the invention is not so limited. For example, in an alternative embodiment, the heel portion may be flat. The heel portion may be generally greater in height from top to bottom relative to the forefoot portion 106. A heel cap may be positioned under the heel portion 102. The heel cap may provide friction. Accordingly, the heel cap may prevent the orthotic shoe insert from sliding excessively in the patent's shoe.

The heel portion 102 may contain the power source 108. The volume of the heel portion 102 may be suitable for housing the power source 108. However, the invention is not so limited. For example, in alternative embodiments, the arch portion or the forefoot portion may contain the power source. The power source 108 may include a battery pack. The battery pack may have sufficient power to power the vibration element 110.

The arch portion 104 may be formed at least partially of an arch fill material. For example, in the embodiment shown, the arch portion 104 may be formed of foam of slightly less density than the material of the heel portion. However, the invention is not so limited. For example, in an alternative embodiment, the arch portion may be formed of vacuum-formed polypropylene. The arch portion 104 may be configured to support the arch of a human foot. The top surface of the arch portion may be contoured so as to correspond with the shape of the arch of the human foot. The arch portion 104 may be generally greater in height from top to bottom relative to the forefoot portion 106. Turning to FIG. 3, the top surface of the arch portion may be sloped from one side to the other when looking at the arch portion 104 from the front. The orthotic shoe insert shown in FIGS. 1-3 is for a left foot. Accordingly, the top surface of the arch portion 104 may slope generally downward from the left side of the orthotic shoe insert (corresponding to the medial side of the patient's left foot) toward the right side of the orthotic shoe insert (corresponding to the lateral side of the patient's left foot). It will be appreciated by those skilled in the art that although the orthotic shoe insert shown in FIGS. 1-3 is for a left foot, the invention is not so limited. An orthotic shoe insert for a right foot may be similarly formed in a generally mirror image of the orthotic shoe insert for the left foot. However, it will be appreciated by those skilled in the art that the exact construction of a particular patient's left orthotic shoe insert may not be an exact mirror image of a right orthotic shoe insert, depending on the patient's size, pathology, feet-type, and feet dimensions.

The arch portion 104 may contain the vibration element 110 and the switch 112. However, the invention is not so limited. For example, in alternative embodiments, the heel portion 102 or the forefoot portion 106 may contain the switch. Although the switch 112 is shown closer to the forefront portion 106 relative to the vibration element 110, the invention is not so limited. For example, in an alternative embodiment, the switch may be positioned between the vibration element and the power source. The switch may be embedded in a vibrating orthotic shoe insert such that the switch is flush with a side surface of the vibrating orthotic shoe insert 100. The switch 112 may be sealed so as to be water resistant or water proof. The switch may be a push button switch. However, the invention is not so limited. For example, in an alternative embodiment, the switch may be a toggle switch.

The vibration element 110 may be located in the arch portion 104 under the porta pedis of a human foot. An example of a vibration element is an unbalanced vibration motor. However, the invention is not so limited. For example, in an alternative embodiment, the vibration element may be a piezoelectric element.

The vibration element 110 may be directed toward the porta pedis. As noted above, the arch portion may be formed at least partially by an arch fill material. At least a sub-portion of the arch portion 104 located above the vibration element may be formed of an arch fill material of a high density.

The higher the density of the fill material, the better the sub-portion of the arch portion 104 may transfer vibration. Accordingly, when power is supplied from the power supply 108 to the vibration element 110 (i.e., the switch is turned on), the vibration element 110 may deliver vibration to the sub-portion to the porta pedis.

The vibration created by the vibration element 110 and delivered to the sub-portion to the porta pedis may be at a force sufficient for pain disruption. However, the vibration may not be audible by a patient when the vibrating orthotic shoe insert is in normal use. The force of the vibration may be a function of the specifications of the power supply and the vibration element. However, the invention is not so limited. In an alternative embodiment, a control circuit may be provided to affect power flowing from the power source to the vibration element thereby controlling the force of the vibration.

The forefoot portion 106 may be formed of a foam of slightly less density than the material of the heel portion. However, the invention is not so limited. For example, in an alternative embodiment, the forefoot portion may be formed of vacuum-formed polypropylene. The forefoot portion 106 may be configured to support the forefoot of a human foot. The forefoot portion 106 may be generally lower in height from top to bottom relative to the arch portion 104 and the heel portion 102.

A method for manufacturing a vibrating orthotic shoe insert according to an embodiment of the present invention is now described with reference FIG. 4 and also with reference to FIGS. 1-3.

In operation 402, dimensions of a vibrating orthotic shoe insert may be set. For example, in an embodiment, the dimensions of a patient's foot may be received. An impression of the patient's foot taken in an impression material may be received. The impression of the patent's foot may be used to set the dimensions of the vibrating orthotic shoe insert.

Accordingly, the dimensions of the vibrating orthotic shoe insert may be custom to the patent's foot. However, the invention is not so limited. For example, in an alternative embodiment, the dimensions of the vibrating orthotic shoe insert may be set according to a standard shoe size. Accordingly, the dimensions of the vibrating shoe insert may be more generic to a number of patients.

In operation 404, the vibrating orthotic shoe insert may be formed according to the set dimensions. The vibrating orthotic shoe insert may include a forefoot portion configured to support a forefoot portion of a human foot. The vibrating orthotic shoe insert may further include an arch portion configured to support an arch of the human foot. The arch portion may include an area configured to be under a porta pedis of the human foot. The vibrating orthotic shoe insert may further include a heel portion configured to support a heel of the human foot. The arch portion may include a vibration element positioned within the area configured to be under the porta pedis of the human foot. The vibration element may be configured to deliver vibration to the porta pedis of the human foot.

According to an embodiment of the present invention, a new protocol for achieving an acceptable level of pain relief for pain associated with certain conditions is provided. This protocol provides multiple advantages over existing protocols. For example, a patient may control when a vibrating orthotic shoe insert is used. Further, a patient may put a vibrating orthotic shoe insert in their own shoe or shoes. Further, a vibrating orthotic shoe insert may be used not only in shoes, but also in, for example, ice-skates, inline skates, boots, and other footwear and athletic equipment.

Alternative and additional features are contemplated to be within the scope of the present invention. For example, cooling mechanisms may be provided to control the temperature of certain features. For example, air channels may be provided to cool the vibration element. Further, an alternative switch may be used. For example, a wireless remote may be used to enable the vibratory element. Those skilled in the art will appreciate these and other modifications and additions.

Although embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A vibrating orthotic shoe insert, comprising:
a forefoot portion configured to support a forefoot portion of a human foot;
an arch portion configured to support an arch of the human foot, the arch portion including an area configured to be under a porta pedis of the human foot; and
a heel portion configured to support a heel of the human foot,
wherein the arch portion comprises a vibration element positioned within the area configured to be under the porta pedis of the human foot, and
wherein the vibration element is configured to deliver vibration to the porta pedis of the human foot.

2. The vibrating orthotic shoe insert of claim 1, wherein the vibration element comprises an unbalanced vibration motor.

3. The vibrating orthotic shoe insert of claim 1, wherein the vibration element is configured to be directed toward the porta pedis of the human foot.

4. The vibrating orthotic shoe insert of claim 1, wherein the arch portion is formed at least partially of an arch fill material and at least a sub-portion of the arch portion located above the vibration element is formed of an arch fill material of a high density.

5. The vibrating orthotic shoe insert of claim 4, wherein the vibration element is configured to deliver vibration to the porta pedis of the human foot through the at least a sub-portion of the arch portion located above the vibration element.

6. The vibrating orthotic shoe insert of claim 1, wherein dimensions of the vibrating orthotic shoe insert correspond to dimensions of a patient's foot.

7. The vibrating orthotic shoe insert of claim 1, wherein the heel portion comprises a power supply for the vibration element.

8. The vibrating orthotic shoe insert of claim 1, further comprising a switch between the power supply and the vibration element.

9. A method of manufacturing a vibrating orthotic shoe insert, comprising:
setting dimensions of the vibrating orthotic shoe insert; and
forming the vibrating orthotic shoe insert according to the set dimensions,
wherein the vibrating orthotic shoe insert includes:
a forefoot portion configured to support a forefoot portion of a human foot;
an arch portion configured to support an arch of the human foot, the arch portion including an area configured to be under a porta pedis of the human foot; and
a heel portion configured to support a heel of the human foot,
wherein the arch portion comprises a vibration element positioned within the area configured to be under the porta pedis of the human foot, and
wherein the vibration element is configured to deliver vibration to the porta pedis of the human foot.

10. The method of manufacturing a vibrating orthotic shoe insert of claim 9, wherein the vibration element comprises an unbalanced vibration motor.

11. The method of manufacturing a vibrating orthotic shoe insert of claim 9, wherein the vibration element is configured to be directed toward the porta pedis of the human foot.

12. The method of manufacturing a vibrating orthotic shoe insert of claim 9, wherein the arch portion is formed at least partially of an arch fill material and at least a sub-portion of the arch portion located above the vibration element is formed of an arch fill material of a high density.

13. The method of manufacturing a vibrating orthotic shoe insert of claim 12, wherein the vibration element is configured to deliver vibration to the porta pedis of the human foot through the at least a sub-portion of the arch portion located above the vibration element.

14. The method of manufacturing a vibrating orthotic shoe insert of claim 9, wherein the setting of the dimensions of the vibrating orthotic shoe includes setting the dimensions of the vibrating orthotic shoe insert according to dimensions of a patient's foot.

15. The method of manufacturing a vibrating orthotic shoe insert of claim 9, wherein the heel portion comprises a power supply for the vibration element.

16. The method of manufacturing a vibrating orthotic shoe insert of claim 9, wherein the vibrating shoe insert further comprises a switch between the power supply and the vibration element.

17. A vibrating orthotic shoe insert, comprising:
a forefoot portion including a top surface configured to support a forefoot portion of a human foot;
an arch portion being formed of an arch fill material and including:
a top surface configured to support an arch of the human foot;
an area configured to be under a porta pedis of the human foot; and
at least a sub-portion of the arch portion located above the area configured to be under the porta pedis of the human foot, the at least a sub-portion being formed of an arch fill material of a high density,
a heel portion including a top surface configured to support a heel of the human foot, the heal portion being formed of a high density material;
a power source positioned within the heal portion; and
a vibration element within the area configured to be under the porta pedis of the human foot within the arch portion, the vibration element configured to receive power from the power source,
wherein the vibration element is configured to deliver vibration to the porta pedis of the human foot through the at least a sub-portion of the arch portion.

18. The vibrating orthotic shoe insert of claim 17, wherein the vibration element comprises an unbalanced vibration motor.
